(19) United States
(12) Patent Application Publication
(10) Pub. No.: US 2025/0049555 A1
(43) Pub. Date: Feb. 13, 2025

(54) STENT, METHOD FOR MANUFACTURING STENT, AND METHOD FOR SECURING AIR FLOW BY RELIEVING STENOSIS OF RESPIRATORY ORGAN

(71) Applicants: TORAY INDUSTRIES, INC., Tokyo (JP); REGENTS OF THE UNIVERSITY OF MINNESOTA, Minneapolis, MN (US)

(72) Inventors: Koji KADOWAKI, Kanagawa (JP); Daniel E. GLUMAC, Lino Lakes, MN (US); Ryan Coulson HUNTER, Little Canada, MN (US); Robroy Henry MACIVER, Minneapolis, MN (US); Gregory Kermit PETERSON, Centerville, MN (US)

(73) Assignees: TORAY INDUSTRIES, INC., Tokyo (JP); REGENTS OF THE UNIVERSITY OF MINNESOTA, Minneapolis, MN (US)

(21) Appl. No.: 18/715,262

(22) PCT Filed: Dec. 1, 2022
(86) PCT No.: PCT/US2022/080726

§ 371 Date: May 31, 2024

Related U.S. Application Data
(60) Provisional application No. 63/285,674, filed on Dec. 3, 2021.

(51) Int. Cl. A61F 2/04; A61L 27/18; A61L 27/58

(52) U.S. Cl. CPC A61F 2/04; A61L 27/18; A61L 27/58; A61F 2002/043; A61F 2002/046; A61F 2210/0004; A61F 2210/0076; A61F 2250/0026; A61F 2250/0036

(57) ABSTRACT

A stent for a respiratory organ includes a bioabsorbable polyester copolymer. The bioabsorbable polyester copolymer is a polyester copolymer having residues of two kinds of ester bond forming monomers as main constituent units, and where the two kinds of ester bond forming monomers are referred to as monomer A and monomer B, respectively, an R value given by the following formula is 0.25 or larger and 0.99 or smaller: R=[AB]/(2[A][B]) 100. In the formula, [A] is a mole fraction (%) of monomer A residues in the polyester copolymer; [B] is a mole fraction (%) of monomer B residues in the polyester copolymer; and [AB] is a mole fraction (%) of structures (A-B and B-A) in which a monomer A residue and a monomer B residue are adjacent to each other in the polyester copolymer.

### TECHNICAL FIELD

The present disclosure relates to a stent for a respiratory organ that contains a bioabsorbable polyester copolymer. The disclosure also relates to a method for manufacturing the stent and a method for securing air flow by relieving stenosis of a respiratory organ using the stent.

### BACKGROUND ART

Stents are implant medical devices that can be left in bodies and some stents can be expanded in the radial direction. Stents are set inside various body cavities or vascular ducts (vascular system, esophagus, gastrointestinal tract, colon and small intestine, bile duct, pancreatic duct, lung pipes, ureter, nasal cavities and trachea, etc.). When a body cavity or a vascular duct is constricted, a stent is set inside a constricted portion to secure an inner cavity.

Among such stents are ones that are left in a body cavity or a vascular duct for a long period of time and ones that are removed from a body after they have been left in a body cavity or a vascular duct and served to keep an inner cavity open for only a prescribed time. For example, Non-patent literature 1 discloses a stent for respiratory tract that is left in a constricted part to enable breathing when the respiratory tract or a bronchus is constricted due to lung cancer, for example.

For stents to be left in a body cavity or a vascular duct for only a prescribed time, there are needs for treatment using a biodegradable polymeric material.

Polylactic acid, polyglycolic acid, polycaprolactone, polydioxanone, and bioabsorbable polyester as a copolymer of these materials are attracting much attention as such bioabsorbable polymeric materials.

For example, although Patent literatures 1 and 2 disclose bioabsorbable stents made of polylactic acid or polycaprolactone, many problems remain that need to be overcome in developing bioabsorbable stents.

### CITATION LIST

### Patent Literature

[Patent literature 1] WO 2020-122096
[Patent literature 2] Japanese Patent No. 6505438

### Non-Patent Literature

[0006][Non-patent literature 1] Yueqi Zhu et al., Materials Today 2017, 20, pp. 516-529.

### SUMMARY

### Technical Problems

Stents are required to be high in the ability to follow a movement of the inside of a body organ because they are required to function properly in an environment in which they are subjected to plural kinds of physical action such as bending, elongation, and pressing when the inside of a body cavity or a vascular duct is moved. Furthermore, in stents for respiratory organs such as a stent for a respiratory tract, occurrence of a complication due to sticking of mucus or a low ability to follow a movement of a body organ is a serious problem. However, the bioabsorbable stents disclosed in Non-Patent literature 1 and Patent literature 2 are low in the ability to follow a movement of a body organ due to their hardness, and hence may move or drop from a set location or hurt neighboring tissue. Furthermore, trocars and catheters for carrying a stent to a part where the stent is to be left are smaller in inner diameter than respiratory tracts. It is difficult to carry a hard stent through a trocar, and hard stents are sometimes damaged. Even if a hard stent is deformed successfully, it is impossible to recover its shape that is suitable for the size of a respiratory tract.

Although Patent literature 1 discloses a medical formed body that contains bioabsorbable polyester and is high in the ability to follow a movement of a body organ, no study is made of a measure against sticking of mucus. Sticking of mucus may cause a complication such as constriction of a stent or an infection.

In view of the above, an object of the present disclosure is to provide a stent for a respiratory organ that can inhibit sticking of mucus because it contains a bioabsorbable polyester copolymer and that is high in the ability to follow a movement of a body organ and thus in biocompatibility.

To attain the above object, the disclosure provides the following:

[1]A stent for a respiratory organ including a bioabsorbable polyester copolymer, in which
the bioabsorbable polyester copolymer is a polyester copolymer having residues of two kinds of ester bond forming monomers as main constituent units; and
where the two kinds of ester bond forming monomers are referred to as monomer A and monomer B, respectively, an R value given by the following formula is 0.25 or larger and 0.99 or smaller:

$$R = [ AB ] / ( 2 [ A ] [ B ] ) \times 100$$

in which:
[A] is a mole fraction (%) of monomer A residues in the polyester copolymer;
[B] is a mole fraction (%) of monomer B residues in the polyester copolymer; and
[AB] is a mole fraction (%) of structures (A-B and B-A) in which a monomer A residue and a monomer B residue are adjacent to each other in the polyester copolymer.

[2] The stent according to item [1], having a Young's modulus as measured in accordance with JIS K6251 (2017) of 0.1 MPa or larger and 50 MPa or smaller.

[3] The stent according to item [1] or [2], having a restorability of 40% or higher, the restorability being defined by the following formula:

$$restorability ( \% ) = ( {L}_{0} \times 2 - {L}_{1} ) / {L}_{0} \times 100$$

in which:
L₀ is an initial length; and
L₁ is a length that is obtained after a manipulation of applying tensile stress to the stent in its longest direction so as to cause tensile strain of 100% based on the initial length L₀ has been performed ten times repeatedly.

[4] The stent according to any one of items [1] to [3], having a mucus sticking amount of 60% or less, the mucus sticking amount being defined by the following formula:

$$mucus sticking amount ( \% ) = ( As - Asb ) \times 100 / ( Ac - Acb )$$

in which:
As is an absorbance at 450 nm of a sample;
Asb is an absorbance at 450 nm of a blank solution for the sample (PBS is used instead of a mucin solution and incubated for one night);
Ac is an absorbance at 450 nm of a stent made of polylactic acid; and
Acb is an absorbance at 450 nm of a blank solution for the stent made of polylactic acid (PBS is used instead of a mucin solution and incubated for one night).

[5] The stent according to any one of items [1] to [4], further including a water-soluble polymer.

[6] The stent according to item [5], having a water-soluble polymer content of 0.1 mass % or more and 25 mass % or less, the water-soluble polymer content being defined by the following formula:

$$water - soluable polymer content ( mass \% ) = [ M 1 / ( M 1 + M 2 ) \times 100 ]$$

in which
M1 is a mass of the water-soluble polymer; and
M2 is a mass of the polyester copolymer.

[7] The stent according to item [5] or [6], in which the water-soluble polymer is a polyalkylene glycol.

[8] The stent according to any one of items [1] to [7], in which the stent contains 50 mass % or more of the bioabsorbable polyester copolymer based on 100 mass % of the stent.

[9] The stent according to any one of items [1] to [8], in which:
the monomer A is at least one selected from the group consisting of lactic acid and glycolic acid; and
the monomer B is at least one selected from the group consisting of caprolactone and δ-valerolactone.

[10] The stent according to any one of items [1] to [9], having an outer diameter of 4 mm or longer and 24 mm or shorter and a thickness of 0.2 mm or larger and 2 mm or smaller.

[11] The stent according to any one of items [1] to [10], having a plurality of projections or a plurality of projections/recesses on an outside surface.

[12] The stent according to item [11], in which the projections or the projections/recesses have a height of 0.1 mm or larger and 3.0 mm or shorter.

[13]A method for manufacturing the stent according to any one of items [1] to [12], including performing 3D printing using a printing material containing the bioabsorbable polyester copolymer.

[14]A method for securing air flow by relieving stenosis of a respiratory organ using a stent for a respiratory organ, in which:
the stent includes a bioabsorbable polyester copolymer,
the bioabsorbable polyester copolymer is a polyester copolymer having residues of two kinds of ester bond forming monomers as main constituent units; and
where the two kinds of ester bond forming monomers are referred to as monomer A and monomer B, respectively, an R value given by the following formula is 0.25 or larger and 0.99 or smaller:

$$R = [ AB ] / ( 2 [ A ] [ B ] ) \times 100$$

in which:
[A] is a mole fraction (%) of monomer A residues in the polyester copolymer;
[B] is a mole fraction (%) of monomer B residues in the polyester copolymer; and
[AB] is a mole fraction (%) of structures (A-B and B-A) in which a monomer A residue and a monomer B residue are adjacent to each other in the polyester copolymer.

The disclosure can provide a stent for a respiratory organ that can inhibit sticking of mucus and that is high in the ability to follow a movement of a body organ and thus high in biocompatibility.

### BRIEF DESCRIPTION OF DRAWINGS

FIG. 1 is a schematic perspective view of a stent according to a specific example of an embodiment;

FIG. 2 is a schematic perspective view of a stent according to another specific example of the embodiment;

FIG. 3 is a schematic perspective view of a stent according to still another specific example of the embodiment;

FIG. 4 is a schematic perspective view of a stent according to yet another specific example of the embodiment;

FIG. 5 is a sectional view, taken along line A-A, of the stent shown in FIG. 1; and

FIG. 6 is a schematic perspective view of a stent according to a further specific example of the embodiment.

### DETAILED DESCRIPTION OF EMBODIMENT

An embodiment will be described below. However, the disclosure is not limited to the following embodiment.

### (Stent)

A stent according to the embodiment is a stent for a respiratory organ that contains a bioabsorbable polyester copolymer.

The term “respiratory organ” as used in the disclosure is a generic term of organs relating to breathing and examples thereof include the respiratory tract, oral cavity, nasal tracts, pharynx, trachea, bronchi, bronchioles, and lungs.

The stent according to the embodiment is a stent for a respiratory organ and is preferably a stent for the respiratory tract, trachea, bronchus, or lung.

Air flow can be secured by relieving stenosis of a respiratory organ by leaving the stent according to the embodiment in a constricted portion of the respiratory organ.

The stent according to the embodiment can be applied to not only a constricted respiratory organ but also a clogged respiratory organ.

### (Bioabsorbable Polyester Copolymer)

The stent according to the embodiment contains a bioabsorbable polyester copolymer.

The stent according to the embodiment is advantageous in that no measure needs to be taken to take out the stent left from a living body since the stent is decomposed in the body after a lapse of a prescribed period because of containing specific bioabsorbable polyester copolymer and resulting substances are subjected to metabolism or discharged from the body. Another advantage is that in the case where part of the stent is decomposed in a living body after a lapse of a prescribed period, the part, remaining in the body, of the stent comes to fit body tissue and hence is not prone to cause a complication.

In the stent according to the embodiment, there are no particular limitations on the content of bioabsorbable polyester copolymer employed as long as the stent contains bioabsorbable polyester copolymer. However, it is preferable that the stent contain 50 mass % or more of bioabsorbable polyester copolymer based on the entire stent (100 mass %), even preferably 80 mass % or more.

If the stent is required to disappear completely when applied to a living body, it is particularly preferable that the stent be composed of bioabsorbable polyester copolymer, that is, contain 100 mass % of bioabsorbable polyester copolymer based on the entire stent (100 mass %). Furthermore, to obtain a stent that is superior in the ability to follow a movement of a body organ as required in the disclosure, it is preferable that the stent contain polyester copolymer described below in the above range.

In the stent according to the embodiment, the term “bioabsorbability” means a property that after being left in or outside a living body a stent decomposes naturally (i.e., biodegrades) by experiencing a hydrolysis reaction or an enzyme reaction and resulting substances disappear because they are subjected to metabolism or discharged from the body.

In the stent according to the embodiment, the polyester copolymer is bioabsorbable.

The polyester copolymer is a copolymer constituted by two or more kinds of monomers including at least one kind of ester bond forming monomer and may be a copolymer having ester bond forming monomer residues as main constituent units.

It is preferable that the polyester copolymer employed in the embodiment contain a polyester copolymer having residues of two or more kinds of ester bond forming monomer as main constituent units and the polyester copolymer may be a polyester copolymer having residues of two kinds of ester bond forming monomer as main constituent units.

In an even preferable aspect of the bioabsorbable polyester copolymer in the stent according to the embodiment, the bioabsorbable polyester copolymer contains a polyester copolymer having residues of two kinds of ester bond forming monomer as main constituent units (in the following description, the polyester copolymer having, as main constituent units, residues of two kinds of ester bond forming monomer contained in such bioabsorbable polyester copolymer may be referred to simply as a “polyester copolymer according to the disclosure.”)

It is preferable that the content of the polyester copolymer be 50 mass % or more based on bioabsorbable polyester copolymer (100 mass %) in the stent, even preferably 70 mass % or more, further preferably 95% or more, and particularly preferably 100 mass %. Setting the content at the above value allows the stent to be restored to an original shape even if it is deformed when receiving plural kinds of physical action such as bending, elongation, and pressing as the inside surface of a body cavity or a vascular duct is moved as well as to inhibit sticking of mucus.

The term “ester bond forming monomer” means a monomer that forms a polymer in which monomer units are connected by ester bonds after polymerization, that is, a monomer that forms a polyester after polymerization.

It is preferable that the ester bond forming monomer be at least one selected from the group consisting of hydroxycarboxylic acids, cyclic esters of hydroxycarboxylic acids, and dimeric cyclic esters of hydroxycarboxylic acids; it is even preferable to use hydroxycarboxylic acids or the like.

That is, it is preferable that the polyester copolymer contain a polyester copolymer having residues of two or more kinds of ester bond forming monomers as main constituent units, and each of the ester bond forming monomer be at least one selected from the group consisting of hydroxycarboxylic acids, cyclic esters of hydroxycarboxylic acids, and dimeric cyclic esters of hydroxycarboxylic acids.

It is particularly preferable to use an aliphatic hydroxycarboxylic acid as a hydroxycarboxylic acid. Examples of aliphatic hydroxycarboxylic acids include lactic acid, glycolic acid, hydroxybutyric acid, hydroxyvaleric acid, hydroxypentanoic acid, hydroxycaproic acid, and hydroxyheptanoic acid, among which lactic acid, glycolic acid, hydroxypentanoic acid, and hydroxycaproic acid are preferable.

Usable examples of lactic acid include L-lactic acid, D-lactic acid, and a mixture thereof. Use of L-lactic acid is preferable from the viewpoint of the physical properties and the biocompatibility of a polymer obtained. In the case where a mixture of L-lactic acid and D-lactic acid is used as the monomer, it is preferable that the content of the L-isomer be 85% or higher, even preferably 95% or higher.

A preferable example of cyclic ester of hydroxycarboxylic acid is lactone which is a cyclic compound obtained through intramolecular dehydration condensation of a hydroxy group and a carboxyl group of a hydroxycarboxylic acid. A preferable example of dimeric cyclic ester of hydroxycarboxylic acid is lactide which is obtained through dehydration condensation of a hydroxy group of one of two molecules of hydroxycarboxylic acid and a carboxyl group of the other.

Usable examples of lactone include caprolactone, dioxepanone, ethylene oxalate, dioxanone, 1,4-dioxane-2,3-dione, trimethylene carbonate, δ-valerolactone, β-propiolactone, β-butyrolactone, γ-butyrolactone, pivalolactone. Caprolactone and δ-valerolactone are particularly preferable.

Usable examples of lactide include dilactide which is obtained through dehydration condensation of two molecules of lactic acid, glycolide which is obtained through dehydration condensation of two molecules of glycolic acid, and tetramethyl glycolide.

Usable examples of the ester bond forming monomer include derivatives of the above-exemplified monomers.

In the embodiment, it is preferable that the ester bond forming monomer be, among the above examples, at least one selected from the group consisting of lactic acid, glycolic acid, hydroxybutyric acid, hydroxyvaleric acid, hydroxypentanoic acid, hydroxycaproic acid, hydroxyheptanoic acid, caprolactone, dioxepanone, ethylene oxalate, dioxanone, 1,4-dioxane-2,3-dione, trimethylene carbonate, δ-valerolactone, β-propiolactone, butyrolactone (β-butyrolactone and γ-butyrolactone), pivalolactone, dilactide, glycolide, and tetramethyl glycolide.

It is preferable that monomer A (described below) be at least one selected from the group consisting of lactic acid, glycolic acid, dilactide, and glycolide; and monomer B (described below) be at least one selected from the group consisting of hydroxyvaleric acid, hydroxycaproic acid, δ-valerolactone, and caprolactone.

It is even preferable that monomer A be lactic acid or glycolic acid and monomer B be caprolactone or δ-valerolactone.

In this specification, in the case where the bioabsorbable polyester copolymer is a polyester copolymer having residues of two kinds of ester bond forming monomer as main constituent units, out of the two kinds of ester bond forming monomers, the monomer that is higher in a crystallinity of a homopolymer consisting of only residues of its monomer is referred to as monomer A and the other monomer that is lower in the crystallinity of a homopolymer consisting of only residues of its monomer is referred to as monomer B. The crystallinity of a homopolymer can be measured using a differential scanning calorimeter (DSC) as mentioned below.

A homopolymer is taken and put on an aluminum pan and subjected to a measurement according to a DSC method using a differential scanning calorimeter (EXTAR 600 produced by Seiko Instruments Inc.) under the following conditions A, and heat of fusion is calculated. A higher heat of fusion per unit mass means higher crystallinity. For example, heat of fusion per unit mass of polylactic acid as measured by this method is 93 J/g.

### (Conditions A)

Instrument name: EXTAR 600 (produced by Seiko Instruments Inc.)
Temperature conditions: from 25° C. to 250° C. (heating rate: 10° C./min)
Standard substance: α-alumina

In the disclosure, it is preferable that the crystallization rates of both of monomer A residues and monomer B residues be lower than 14%. If these crystallization rates are lower than 14%, increase of the Young's modulus can be inhibited such that a polyester copolymer that is suitable for stents can be obtained. It is preferable that the crystallization rates of monomer A residues and monomer B residues be 10% or lower, even preferably 5% or lower.

The crystallization rate of residues of a certain monomer is the ratio of heat of fusion per unit mass of residues of this monomer in a polyester copolymer employed in the disclosure to the product of heat of fusion per unit mass of a homopolymer consisting of only residues of this monomer and a mass fraction of the residues of this monomer in the polyester copolymer employed in the disclosure. That is, the crystallization rate of residues of monomer A is the ratio of heat of fusion per unit mass of residues of monomer A in a polyester copolymer employed in the disclosure to the product of heat of fusion per unit mass of a homopolymer consisting of only monomer A and a mass fraction of the residues of monomer Ain the polyester copolymer employed in the disclosure. The crystallization rate of residues of monomer A or monomer B indicates a proportion of residues having crystal structures in the residues of monomer A or monomer B.

In particular, in the case where the monomer A residues are lactic acid residues and the monomer B residues are caprolactone residues, it is preferable that the crystallization rates of the lactic acid residues and the caprolactone residues be smaller than 14%, even preferably 10% or smaller. A crystallization rate is determined in accordance with the following specific method.

A polyester copolymer is dissolved in chloroform so that its concentration becomes 5 mass %, and the resulting solution is moved to a Teflon (registered trademark) petri dish and dried for 20 to 24 hours at normal pressure and room temperature (20° C. to 25° C.). A resulting substance is subjected to drying under reduced pressure, and thus a polyester copolymer film is obtained. The obtained polyester copolymer film is put in an alumina pan, subjected to a measurement according to a DSC method using a differential scanning calorimeter under the following conditions, and heat of fusion is calculated from measurement results of temperature conditions (D) and (E). A crystallization rate is calculated according to the following formula.

$$Crystallization rate of lactic acid residues ( \% ) = \{ ( heat of fusion per unit mass of lactic acid residues in polyester copolymer ) / ( heat of fusion per unit mass of homopolymer consisting of only lactic acid residues ) \times ( mass fraction of lactic acid residues in polyester copolymer ) \} \times 100$$

$$Crystallization rate of caprolactone residues ( \% ) = \{ ( heat of fusion per unit mass of caprolactone residues in polyester copolymer ) / ( heat of fusion per unit mass of homopolymer consisting of only caprolactone residues ) \times ( mass fraction of caprolactone residues in polyester copolymer ) \} \times 100$$

Instrument name: EXTAR 600 (produced by Seiko Instruments Inc.)
Temperature conditions: (A) 25° C.->(B) 250° C. (heating rate: 10° C./min)->(C) 250° C. (holding time: 5 min)->(D)-70° C. (cooling rate: 10° C./min)->(E) 250° C. (heating rate: 10° C./min)->(F) 250° C. (holding time: 5 min)->(G) 25° C. (cooling rate: 100° C./min)
Standard substance: alumina

In this specification, as a general rule, the term “monomer residue” means a unit of repetition of a chemical structure originating from the monomer in a chemical structure of a copolymer obtained by polymerizing two or more kinds of monomers including the monomer concerned. For example, in the case where a copolymer of lactic acid and caprolactone is produced by polymerizing lactic acid (CH₃CH(OH)COOH) and caprolactone (ε-caprolactone, see the following formula),

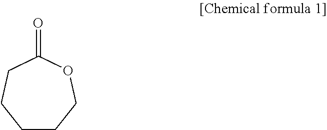

a lactic acid monomer residue is represented by the following formula

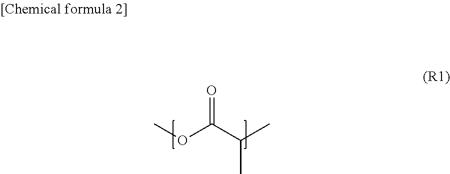

and a unit represented by the following formula

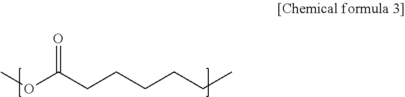

is a caprolactone monomer residue.

As an exception, in the case where a dimer such as a lactide is used as a monomer, the term “monomer residue” means one of two repetitive structures originating from the dimer. For example, in the case where dilactide (L-(-)-lactide, see the following formula)

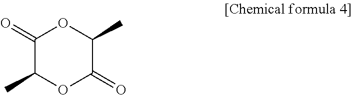

and caprolactone are polymerized, the chemical structure of a resulting copolymer has, as a dilactide residue, a repetitive structure consisting of two structures each represented by the above formula (R1). In this case, one of the two lactic acid units is regarded as a “monomer residue” and it is considered that two monomer residues, that is, two lactic acid residues, are formed originating from dilactide.

The bioabsorbable polyester copolymer of this embodiment is a polyester copolymer having, as main constituent units, residues of two kinds of ester bond forming monomers. Here, having two kinds of monomer residues as “main constituent units” means that the sum of the number of two kinds of monomer residues is 50 mol % or larger, and the number of each of the two kinds of monomer residues is 20 mol % or larger when the sum of the numbers of all kinds of monomer residues, including other kinds of monomer residues, contained in the entire polymer is regarded as 100%. For example, “having a monomer A residue and a monomer B residue as main constituent units” means that the sum of the number of monomer A residues and the number of monomer B residues is 50 mol % or larger, the number of monomer A residues is 20 mol % or larger, and the number of monomer B residues is 20 mol % or larger when the sum of the numbers of all kinds of monomer residues contained in the entire polymer is regarded as 100%.

A mole fraction of residues of each of monomer A, monomer B, and other kinds of monomers can be determined from an area value of a signal originating from each kind of residues obtained by a nuclear magnetic resonance (NMR) measurement. For example, in the case where monomer A residues are lactic acid residues and monomer B residues are caprolactone residues, mole fractions of them can be measured by a method to be described later in Measurement Example 1.

According to the above definition, the sum of the number of monomer A residues and the number of monomer B residues is 50 mol % or larger when the sum of the numbers of all kinds of monomer residues, including other kinds of monomer residues, contained in the entire polymer is regarded as 100%, preferably 75 mol % or larger and even preferably 90 mol % or larger. Also, according to the above definition, the number of monomer A residues and the number of monomer B residues each are 20 mol % or larger, preferably 30 mol % or larger and even preferably 40 mol % or larger. A polymer in which the sum of the number of monomer A residues and the number of monomer B residues amounts to 100 mol % (entire polymer), that is, a polymer consisting of only monomer A and monomer B, is a particularly preferable aspect.

It is possible to further allow another monomer that can copolymerize with the two kinds of ester bond forming monomers constituting main constituent units to copolymerize as long as the advantages of the disclosure are not impaired. Another of the above-mentioned example ester bond forming monomers can be used as such a monomer.

It is also preferable to cause copolymerization with a monomer that functions as a linker. Examples of a monomer that functions as a linker include hydroxycarboxylic acid other than two kinds of ester bond forming monomers that constitute main constituent units, dialcohol, dicalbonic acid, amino acid, diamine, diisocyanate, and diepoxide.

In this specification, the term “polyester copolymer” encompasses also copolymers containing a monomer other than ester bond forming monomers and thus containing a constituent unit that is connected by a bond other than an ester bond.

The polyester copolymer in the embodiment needs to be bioabsorbable. Those skilled in the art would be able to synthesize a copolymer that exhibits suitable bioabsorbability for a use by combining appropriate monomers exemplified above and adjusting the quantity ratio of the monomers within a range specified in the disclosure.

With respect to the mole ratio between monomer A residues and monomer B residues in the polyester copolymer in the disclosure, if one of polymer A and polymer B exists excessively, the polyester copolymer comes to exhibit properties that are close to properties of a homopolymer. Thus, the ratio of the mole number of monomer A residues to the total of those of monomer A residues and monomer B residues (total mole number, 100%) is preferably 20% to 80%, even preferably 30% to 70% and further preferably 40% to 60%.

In the stent according to the embodiment, it is preferable that the polyester copolymer be a polyester copolymer having residues of two kinds of ester bond forming monomers as main constituent units, and where out of the two kinds of ester bond forming monomers, the monomer that is higher in a crystallinity of a homopolymer consisting of only its monomer residues is referred to as monomer A and the other monomer that is lower in the crystallinity of a homopolymer consisting of only its monomer residues is referred to as monomer B, an R value given by the following formula be 0.25 or larger and 0.99 or smaller.

$$R = [ AB ] / ( 2 [ A ] [ B ] ) \times 100$$

in which:
[A] is a mole fraction (%) of monomer A residues in the polyester copolymer;
[B] is a mole fraction (%) of monomer B residues in the polyester copolymer; and
[AB] is a mole fraction (%) of structures (A-B and B-A) in which a monomer A residue and a monomer B residue are adjacent to each other in the polyester copolymer.

The R value is used as an index indicating the degree of randomness of an arrangement of residues of two kinds of ester bond forming monomers, that is, monomer residues of a copolymer having a monomer A residue and monomer B residue as main constituent elements. For example, a random copolymer in which the arrangement of monomer residues is completely random has an R value of 1.

An R value can be determined by quantifying a ratio of the number of combinations A-A, B-B, A-B, and B-A to the number of combinations of two adjacent monomer residues (hereinafter may be referred to as “dyads”) by a nuclear magnetic resonance (NMR) measurement, and, more specifically, measured by a method to be described later in Measurement Example 1. For example, in the case where a polyester copolymer consists of only monomer A and monomer B, [AB] means the ratio of the sum of the number of A-B dyads and the number of B-A dyads to the number of all dyads (A-A, B-B, A-B, and B-A) in the polyester copolymer.

In the case where three or more kinds of ester bond forming monomers exist, two kinds of ester bond forming monomers contained most are selected, and out of the two, a monomer that is higher in the crystallinity of a homopolymer consisting of only its monomer residues is referred to as monomer A and the other monomer that is lower in the crystallinity of a homopolymer consisting of only its monomer residues is referred to as monomer B.

For example, in the case where a polyester copolymer consists of three kinds of monomers, that is, monomer X, monomer Y, and monomer Z, two kinds of monomers having higher contents contained most are selected, and out of these two kinds of monomers, the monomer that is higher in the crystallinity of a homopolymer is referred to as monomer A and the other monomer that is lower in the crystallinity of a homopolymer is referred to as monomer B. A monomer contained least is referred to as monomer C. In this case, [AB] means the ratio of the sum of the number of A-B dyads and the number of B-A dyads to the number of all dyads (A-A, B-B, A-B, B-A, A-C, C-A, B-C, C-B, and C-C) in the polyester copolymer. A similar procedure can be applied in the case where a polyester copolymer consists of four or more kinds of monomers.

When the R value is smaller than 0.25, the polyester copolymer may be high in crystallinity and a stent obtained may be hard, as a result of which its Young's modulus becomes large and its restorability lowers. On the other hand, when the R value is larger than 0.99, a stent obtained may be so soft as to be adhesive, and the stent may be difficult to handle or may be low in restorability. Thus, to exhibit a high ability to follow a movement of a body organ and high biocompatibility by inhibiting sticking of mucus, it is preferable that the R value be 0.25 or larger and 0.99 or smaller, more preferably 0.45 or larger and 0.99 or smaller, even preferably 0.50 or larger and 0.85 or smaller, and further preferably 0.50 or larger and 0.80 or smaller.

To control the crystallization rate in a proper range, it is preferable that the weight-average molecular weight of the polyester copolymer in the embodiment be in a range of 80,000 to 1,000,000, more preferably in a range of 100,000 to 1,000,000, further preferably in a range of 120,000 to 750,000, even preferably in a range of 150,000 to 750,000, still further preferably in a range of 150,000 to 500,000, and particularly preferably in a range of 200,000 to 500,000. For example, a weight-average molecular weight of a polyester copolymer can be measured by a method described in Measurement Example 2.

### (Method for Manufacturing Polyester Copolymer)

For example, a polyester copolymer in the embodiment can be manufactured by a polyester copolymer manufacturing method including:
a macromer synthesizing step of blending and polymerizing two kinds of ester bond forming monomers, that is, monomer A and monomer B, such that at the completion of polymerization the sum of the number of monomer A residues and the number of monomer B residues becomes larger than or equal to 50 mol % of the number of all residues, the number of monomer A residues becomes larger than or equal to 20 mol % of the number of all residues, and the number of monomer B residues becomes larger than or equal to 20 mol % of the number of all residues; and
a multiplying step of multiplying macromers obtained in the macromer synthesizing step by connecting the macromers to each other or adding monomers A and monomers B to a macromer solution obtained by the macromer synthesizing step.

### (Macromer Synthesizing Step)

In the macromer synthesizing step, monomer A and monomer B are blended and polymerized so that at the completion of the polymerization, theoretically, the sum of the number of monomer A residues and the number of monomer B residues becomes larger than or equal to 50 mol % of the number of all residues, the number of monomer A residues becomes larger than or equal to 20 mol % of the number of all residues, and the number of monomer B residues becomes larger than or equal to 20 mol % of the number of all residues. As a result, a polyester copolymer having monomer A residues and monomer B residues as main constituent units. Since the multiplying step (described later) is performed further in this manufacturing method, in this specification the polyester copolymer obtained by this step is referred to as a “macromer.”

The same ester bond forming monomers as exemplified above can be used, and preferable combinations of ester bond forming monomers and other items follow the related descriptions made above.

The degree of randomness of the distribution of monomer residues constituting a polyester copolymer having two kinds of ester bond forming monomers as main constituent units varies depending on reactivities of the monomers during polymerization. That is, a copolymer in which monomer residues are distributed completely randomly is obtained if, during polymerization, one of the two kinds of monomers and the other are combined with a monomer from behind at the same probability. However, if either kind of monomer tends to be combined more easily with one kind of monomer from behind, a gradient copolymer is obtained in which the distribution of monomer residues is uneven. In the thus-obtained gradient copolymer, the composition of a monomer residue varies continuously from a polymerization starting end to a polymerization finishing end along a molecular chain.

If monomer A is higher in initial polymerization rate than monomer B, when monomers A are copolymerized with monomers B in the macromer synthesizing step monomer A tends to be combined with monomer A from behind. As a result, a synthesized macromer is given a gradient structure having a composition gradient in which the proportion of a monomer A unit decreases gradually as the position goes from a polymerization starting end to a polymerization finishing end. That is, a macromer obtained by this step is given a gradient structure in which monomer A residues and monomer B residues are arranged in the backbone so as to produce a composition gradient. That is, in this step, use of monomer A and monomer B that are different from each other in initial polymerization rate makes it possible to produce a macromer having a gradient structure in which the backbone has a composition gradient. In this specification, such a macromer may be referred to as a “gradient macromer.”

In the macromer synthesizing step, to realize such a gradient structure, it is desirable to synthesize a macromer by a polymerization reaction that occurs in one direction from a starting end. Preferable examples of such a synthesizing reaction include reactions that utilize ring-opening polymerization or living polymerization.

To facilitate manufacture of a polyester copolymer that satisfy the above R value range finally, it is preferable that a macromer produced in this step have an R′ value that is similar to an intended R value of a polyester copolymer, that is, an R′ value that is given by the following formula be 0.25 or larger and 0.99 or smaller:

$${R}^{′} = [ {AB}^{′} ] / ( 2 [ {A}^{′} ] [ {B}^{′} ] ) \times 100$$

in which
[A′] is a mole fraction (%) of monomer A residues in the macromer;
[B′] is a mole fraction (%) of monomer B residues in the macromer; and
[AB′] is a mole fraction (%) of structures (A′-B′ and B′-A′) in which a monomer A residue and a monomer B residue are adjacent to each other in the macromer.

It is preferable that the weight-average molecular weight of a macromer synthesized in the macromer synthesizing step be 10,000 or larger, even preferably 20,000 or larger. To keep a macromer flexible while setting its crystallinity low, it is preferable that the weight-average molecular weight of a macromer synthesized in the macromer synthesizing step be 150,000 or smaller, even preferably 100,000 or smaller.

### [Multiplying Step]

In the multiplying step, multiplying is performed by connecting macromers obtained by the macromer synthesizing step or adding monomers A and monomers B to a macromer solution obtained by the macromer synthesizing step. This step may be executed either by connecting macromers obtained by one macromer synthesizing step or connecting plural macromers obtained by two or more macromer synthesizing steps. The term “multiplying” means forming a structure in which plural molecular chains are repeated each having a gradient structure in which monomer A residues and monomer B residues are arranged in the backbone so as to produce a composition gradient.

The number of macromer units to be multiplied may be 2 or more. Since the effect of increasing the tensile strength is obtained by entanglement of molecular chains when the number of connections is large, it is preferable that the number of macromer units to be multiplied be 3 or larger, even preferably 4 or larger and further preferably 6 or larger. On the other hand, since if the molecular weight of a manufactured polyester copolymer becomes too large, a resulting increase in viscosity may adversely affects the formability, it is preferable that the number of macromer units be 80 or smaller, even preferably 40 or smaller and further preferably 20 or smaller.

The number of connections of macromer units can be adjusted by a catalyst used in the multiplying step and a reaction time. When macromers are multiplied by connecting them, the number of macromer units can be calculated by dividing a weight-average molecular weight of a finally obtained polyester copolymer by a weight-average molecular weight of the macromers.

Example catalysts that can be used in the multiplying step include 4-(dimethylamino)pyridinium-p-toluenesulfonate and 4-dimethylaminopyridine.

The polyester copolymer in the disclosure may be either a straight-chain polymer in which macromer units are connected to each other linearly or a branched-chain polymer in which macromer units are connected to have a branched chain.

For example, a straight-chain polyester copolymer can be synthesized by combining a gradient macromer with a similar gradient macromer by connecting ends of the gradient macromers one molecule at a time.

In the case where a gradient macromer has a hydroxyl group and a carboxyl group at each end, a multiplied polyester copolymer can be obtained by causing condensation at their ends using a condensing agent. Usable examples of the condensing agent include 4-(dimethylamino)pyridinium-p-toluenesulfonate, 1-[3-(dimethylamino)propyl]-3-ethylcalbodiimide, hydrochloric acid 1-ethyl-3-(3-dimethylaminopropyl)carbodiimide, N, N′-dicyclohexylcarbodiimide, N, N′-diisopropylcarbodiimide, N, N′-carbonyldiimidazole, 1,1′-calbonyldi(1,2,4-triazole), 4-(4,6-dimethoxy-1,3,5-triazin-2-yl)-4-methylmorpholinium chloride n-hydrate, (4,6-dimethoxy-1,3,5-triazin-2-yl)-(2-octoxy-2-oxoethyl)dimethylammonium trifluoromethanesulfonate, 1H-benzotriazol-1-yloxytris(dimethylamino)phosphonium hexafluorophosphate, 1H-benzotriazol-1-yloxytripyrrolidino phosphonium hexafluorophosphate, (7-azabenzotriazol-1-yloxy)tripyrrolidino phosphonium hexafluorophosphate, chlorotripyrrolidino phosphonium hexafluorophosphate, bromotris(dimethylamino)phosphonium hexafluorophosphate, 3-(diethoxyphosphoryloxy)-1, 2, 3-benzotriazin-4(3H)-one, O-(benzotriazol-1-yl)-N, N, N′,N′-tetramethyluronium hexafluorophosphate, 0-(7-azabenzotriazol-1-yl)-N, N, N′,N′-tetramethyluronium hexafluorophosphate, O-(N-succinimidyl)-N, N, N′,N′-tetramethyluronium tetrafluoroborate, O-(N-succinimidyl)-N, N, N′,N′-tetramethyluronium hexafluorophosphate, O-(3,4-dihydro-4-oxo-1, 2, 3-benzotriazin-3-yl)-N, N, N′,N′-tetramethyluronium tetrafluoroborate, S-(1-oxide-2-pyridyl)-N, N, N′,N′-tetramethyluronium tetrafluoroborate, 0-[2-oxo-1(2H)-pyridyl]-N, N, N′,N′-tetramethyluronium tetrafluoroborate, {{[(1-cyano-2-ethoxy-2-oxoethylidene)amino]oxy}-4-morpholinomethylene}dimethylammonium hexafluorophosphate, 2-chloro-1,3-dimethylimidazolinium hexafluorophosphate, 1-(chloro-1-pyrrolidinylmethylene)pyrrolidinium hexafluorophosphate, 2-fluoro-1,3-dimethylimidazolinium hexafluorophosphate, and fluoro-N, N, N′,N′-tetramethylformamidinium hexafluorophosphate.

In the case where a polymerization reaction has a certain degree of livingness, that is, where a polymerization reaction can start from an end of a polymer and continue, multiplying can be performed by repeating a manipulation of adding monomer A and monomer B to a gradient macromer solution after completion of the polymerization reaction.

Alternatively, gradient macromers may be multiplied via a linker in such a range that no influences are exerted on the kinetic properties of the polymer. In particular, use of a linker having plural carboxyl groups or plural hydroxy groups, such as 2,2-bis(hydroxymethyl)propionic acid, makes it possible to synthesize a branched polyester copolymer in which the linker serves as a branching point.

A polyester copolymer obtained by the above-described manufacturing method is a copolymer having a structure where two or more macromer units in each of which monomer A residues and monomer B residues are arranged so as to produce a composition gradient in the backbone are connected to each other. This is a preferable aspect of the polyester copolymer in the disclosure. In this specification, for the sake of convenience, such a structure may be referred to as a “multigradient” structure and a copolymer having a multigradient structure may be referred to as a “multigradient copolymer.”

That is, it is preferable that the polyester copolymer in the disclosure be a multigradient copolymer. It is preferable that the multigradient copolymer have a structure where two or more macromer units having a gradient structure in which monomer A residues and monomer B residues exhibit a composition gradient in the backbone, even preferably a structure that three or more such macromer units are connected to each other. As for the upper limit of the number of connections of macromer units having a gradient structure in which monomer A residues and monomer B residues exhibit a composition gradient in the backbone, it is preferable that the number of connections of such macromer units may be 80 or smaller, even preferably 40 or smaller and further preferably 20 or smaller.

As described above, a polyester copolymer in which the monomer A residue is a lactic acid and the monomer B residue is a caprolactone residue is particularly preferable. Such a polyester copolymer can be manufactured preferably by the following manufacturing method.

First, in a macromer synthesizing step, dilactide and ε-caprolactone are polymerized with each other under the presence of a catalyst. Preferably, dilactide and ε-caprolactone monomers are refined before use to remove impurities. For example, dilactide can be refined by recrystallization from toluene that has been dried using potassium. For example, ε-caprolactone is refined by vacuum distillation from CaH₂ in an N₂ atmosphere.

Usable examples of a catalyst to be used in the macromer synthesizing step for polymerizing a macromer having lactic acid residues and caprolactone residues include ordinary polyester polymerization catalysts such as germanium-based, titanium-based, antimony-based, and tin-based catalysts. Specific examples of these polyester polymerization catalysts include tin octylate, antimony trifluoride, a zinc powder, dibutyltin oxide, and tin oxalate. There are no particular limitations on the method for adding a catalyst to a reaction system. However, it is preferable to employ a method of adding a catalyst when the raw materials are prepared in a state where the catalyst is dispersed in raw materials or adding a catalyst at a start of pressure reduction in a state where the catalyst has been subjected to dispersion processing. It is preferable that the amount of a catalyst used be 0.01 to 3 mass % (in terms of metal atoms) based on the entire amount of monomers used, even preferably 0.05 to 1.5 mass %.

A macromer having lactic acid residues and caprolactone residues can be obtained by putting dilactide, caprolactone, and a catalyst into a reaction container that is equipped with a stirrer and allowing dilactide and caprolactone to react with each other at 120° C. to 250° C. in a nitrogen gas flow.

The reaction may be caused using an initiator such as hydroxypivalic acid, alcohol, or the like. In the case where water is used as an auxiliary initiator, it is preferable to cause an auxiliary catalyzation reaction at about 90° C. prior to a polymerizing reaction.

The reaction time may be 2 hours or longer, preferably 4 hours or longer. To increase the degree of polymerization further, the reaction time is preferably further longer such as 8 hours. However, since too long a reaction time may incur a coloration problem of polymer, a reaction time of 3 to 30 hours is preferable.

Subsequently, in a multiplying step, multiplying is performed by connecting ends of gradient macromers having lactic acid residues and caprolactone residues by a condensation reaction. It is preferable that the reaction temperature of the condensation reaction be 10° C. to 100° C., even preferably 20° C. to 50° C. It is preferable that the reaction time be longer than or equal to one day, even preferably longer than or equal to two days. However, since too long a reaction time may incur a coloration problem of polymer, a reaction time of two to four days is preferable.

It is preferable that the polyester copolymer in the embodiment have a structure where two or more macromer units are connected to each other, and where the macromer unit is a polyester copolymer having, as main constituent units, monomer A residues and monomer B residues that satisfy 1.1≤V_{X}/V_{Y}≤40 in which V_{X} is a higher one of initial polymerization rates of monomer A and monomer B and V_{Y} is a lower one.

When a polyester copolymer is produced which has a structure where two or more macromer units are connected to each other, the macromer unit being a polyester copolymer having, as main constituent units, monomer A residues and monomer B residues that satisfy 1.1≤V_{X}/V_{Y}≤40, a macromer unit having a gradient structure can be obtained. As a result, the polyester copolymer in the embodiment is given a multigradient structure, which is preferable.

In this specification, the term “macromer” means a polyester copolymer that is obtained by the above-described macromer synthesizing step and since it is a polyester copolymer to be used in the above-described multiplying step after the macromer synthesizing step, the polyester copolymer is referred to as a “macromer” to avoid confusion. The term “macromer unit” means a portion made up of one macromonomer in a polyester copolymer molecular chain. For example, in the case where a polyester copolymer is formed by connecting two macromers, this polyester copolymer is a polyester copolymer having a structure where two macromer units are connected to each other.

The expression “two kinds of monomer residues are ‘main constituent units’ of a macromer unit” means that the sum of the numbers of these two kinds of monomer residues is larger than or equal to 50 mol % of the sum of the numbers of all kinds of monomer residues, including other kinds of monomer residues, contained in the entire macromer unit (100%) and that the number of each kind of residues is larger than or equal to 20 mol % of the sum of the numbers of all kinds of monomer residues contained in the entire macromer unit (100%). For example, the expression “a monomer A residue and a monomer B residue are main constituent units” means that the sum of the numbers of these two kinds of monomer residues is larger than or equal to 50 mol % of the sum of the numbers of all kinds of monomer residues contained in the entire macromer unit (100%), that the number of monomer A residues is larger than or equal to 20 mol %, and that the number of monomer B residues is larger than or equal to 20 mol %. Mole fractions of monomer A residues, monomer B residues, and other kinds of monomer residues can be determined by a nuclear magnetic resonance (NMR) measurement from an area value of a signal originating from each kind of residues. For example, in the case where monomer A residues are lactic acid residues and monomer B residues are caprolactone residues, mole fractions of them can be measured by a method to be described later in Measurement Example 1.

V_{X} and V_{Y} that are a higher one and a lower one, respectively, of initial polymerization rates of monomer A and monomer B are determined by the following method. Equimolar amounts of monomer A and monomer B are blended, a solvent or a catalyst is added if necessary, and a polymerization reaction is started under conditions such as a temperature adjusted such that the R value of a polyester copolymer finally synthesized or intended to be synthesized becomes equal to the above-mentioned value within an error range of 10%. A sample being polymerized is subjected to sampling regularly to measure residual amounts of monomer A and monomer B. A residual amount is measured by chromatography or a nuclear magnetic resonance (NMR) measurement, for example. A monomer amount that has been used for the polymerization reaction is determined by subtracting a residual amount from a prepared amount. An initial gradient of a curve obtained by plotting monomer amounts subjected to the polymerization reaction with respect to sampling times is V_{X} or V_{Y}.

If monomer A is higher in initial polymerization rate than monomer B, when such monomer A and monomer B are caused to react with each other, monomer A is connected to an end of a polymer under polymerization at a high probability in an initial part of the polymerization. On the other hand, in a later part of the polymerization in which the concentration, in a reaction liquid, of monomer A that has been consumed earlier is low, the probability that monomer B is connected to an end of a polymer under polymerization is high. As a result, a gradient polymer is obtained in which the proportion of monomer A residues decreases gradually from its one end. Such a gradient polymer is low in crystallinity and the Young's modulus is less likely to increase. To facilitate formation of such a gradient structure, it is preferable that V_{X}/V_{Y} be 1.3 or larger, even preferably 1.5 or larger. On the other hand, if the difference between the polymerization rates of monomer A and monomer B is too large, a gradient polymer becomes, in structure, similar to a block polymer in which monomers B are polymerized after only monomers A has been polymerized, possibly resulting in high crystallinity and increase in Young's modulus. Thus, it is preferable that V_{X}/V_{Y} be 30 or smaller, even preferably 20 or smaller and further preferably 10 or smaller.

Preferable Example combinations of monomer A and monomer B as described above include dilactide and ε-caprolactone, glycolide and ε-caprolactone, glycolide and dilactide, dilactide and dioxepanone, ethylene oxalate and dilactide, dilactide and δ-valerolactone, and glycolide and δ-valerolactone.

As mentioned above, it is preferable that the stent according to the embodiment contain, as a bioabsorbable polyester copolymer, a polyester copolymer having residues of two kinds of ester bond forming monomers as main constituent units. It is also preferable that the stent according to the embodiment further contain a water-soluble polymer that is different from bioabsorbable polyester copolymer employed. There are no particular limitations on the content of the water-soluble polymer that is different from the bioabsorbable polyester copolymer. However, it is preferable that the stent according to the embodiment contain a water-soluble polymer that is different from the bioabsorbable polyester copolymer in an amount of 0.1 to 50 mass %, even preferably 0.1 to 10 mass %.

In addition, from the view point of suppressing sticking of mucus, the stent according to the embodiment preferably has a water-soluble polymer content defined by the following formula being 0.1 mass % or more and 25 mass % or less, more preferably 1.0 mass % or more and 10 mass % or less, and further preferably 2.0 mass % or more and 5.0 mass % or less.

$$Water - soluable polymer content ( mass \% ) = [ M 1 / ( M 1 + M 2 ) \times 100 ]$$

M1 is a mass of the water-soluble polymer
M2 is a mass of the polyester copolymer

The water-soluble polymer that is different from the bioabsorbable polyester copolymer is a polymer that is other than a bioabsorbable polyester copolymer and can dissolve in water. Whether a polymer falls into this category can be judged specifically by determining whether it dissolves in water by a measurement method described later.

The water-soluble polymer consists of a water-soluble material. However, the water-soluble polymer may contain additives etc. other than the above material as long as they do not impair manifestation of water-solubility. A material (polymer) is judged “water soluble” if dissolution of a polymer is found visually when 1 g of the polymer is put in 100 mL of water heated to 37° C. and then the water is stirred for 3 hours. It is preferable that the water-soluble material be a material that dissolves by 1 part by weight in 100 parts by weight of water at 37° C.

Examples of the water-soluble polymer that is different from the bioabsorbable polyester copolymer include polyalkyleneglycol (e.g., polyethyleneglycol and polypropyleneglycol), polyacrylic acid, polymethacrylic acid, polyacrylamide, polyvinylpyrrolidone, polyvinylacetoamide, polymaleic acid, polysulfonic acid, polyvinyl alcohol, polyethyleneimine, calboxymethyl cellulose, alginic acid, polyphosphoric acid, starch, agar, gelatin, pullulan, dextrin, and xanthan gum and their salts, copolymers, and copolymer salts. Among these examples, polyalkyleneglycol, polyacrylic acid, polymethacrylic acid, polyacrylamide, polyvinylpyrrolidone, and polyvinyl alcohol and their salts, copolymers, and copolymer salts are preferable. Polyalkyleneglycol and polyvinyl alcohol and their salts, copolymers, and copolymer salts are even preferable. Polyalkyleneglycol is most preferable.

As described above, it is preferable that the stent according to the embodiment contain, as a bioabsorbable polyester copolymer, a polyester copolymer having residues of two kinds of ester bond forming monomers as main constituent units. It is also preferable that the stent further contain a bioabsorbable homopolymer. That is, a stent that contains, as bioabsorbable polyester copolymers, both of a polyester copolymer having residues of two kinds of ester bond forming monomers as main constituent units and a homopolymer is a preferable aspect of the stent according to the embodiment.

That is, the stent according to the embodiment may contain, in addition to a bioabsorbable polyester copolymer, polyester other than the bioabsorbable polyester copolymer.

Examples of such polyester other than the bioabsorbable polyester copolymer include a polyester selected from the group consisting of polyglycolic acid, polylactic acid (D, L, and DL types), poly(ε-caprolactone), polyhydroxybutylate, polyhydroxybutylate-valerate, polyorthoester, polyhydroxyvalerate, polyhydroxyhexanoate, polybutylenesuccinate, polytrimethylenete telephthalate, polyhydroxy alkanoate, and polydioxanone.

It is preferable that the stent according to the embodiment contain a copolymer of lactic acid and glycolic acid or a copolymer of lactic acid and ε-caprolactone. The stent according to the embodiment may further contain polylactic acid.

In the case where the stent according to the embodiment contains, as bioabsorbable polyester copolymers, both of a polyester copolymer having residues of two kinds of ester bond forming monomers as main constituent units and a homopolymer, to maintain the ability to follow a movement of a body organ, it is preferable that the content of the homopolymer in the stent be 50 mass % or lower in bioabsorbable polyester copolymers (100 mass %), even preferably 30 mass % or lower. Although there are no particular limitations on its lower limit, the content of the homopolymer in the stent is preferably 1 mass % or higher.

It is preferable that a Young's modulus of the stent according to the embodiment as measured in accordance with JIS K6251 (2017) be 0.1 MPa or larger and 50 MPa or smaller. This measurement method is as described in Measurement Example 3 later. Stents are left in various kinds of body cavities or vascular ducts. Thus, if the Young's modulus of a stent is too large, when the stent receives external force produced by its deformation such as bending or curving, the stent may damage tissue around the position where the stent is left by, for example, pressing, scratching, or sticking the tissue. It is therefore preferable that the Young's modulus of a stent be 50 MPa or smaller, even preferably 20 MPa or smaller and further preferably 10 MPa or smaller.

On the other hand, if the Young's modulus of a stent is too small, when the stent receives external force produced by its deformation such as bending or curving the stent may no longer maintain its shape. It is therefore preferable that the Young's modulus of a stent be 0.1 MPa or larger, even preferably 0.5 MPa or larger and further preferably 1.0 MPa or larger.

It is preferable that tensile strength of the stent according to the embodiment as measured in accordance with JIS K6251 (2017) be 4 MPa or larger. This measurement method is as described in Measurement Example 3 later. The tensile strength is a factor that closely relates to the fracture resistance strength of a stent. Thus, it is preferable that the tensile strength of a stent be 4 MPa or larger and more preferably 5 MPa or larger. In the case of a stent to be set in a part where a large deformation such as bending or curving may occur, it is preferable that its tensile strength be 10 MPa or larger, even preferably 15 MPa or larger. It is preferable that the tensile strength of a stent may be as high as possible. Although there are no particular limitations on the upper limit of the tensile strength, a realistic upper limit of the tensile strength would be about 500 MPa.

It is preferable that elongation at break of the stent according to the embodiment as measured in accordance with JIS K6251 (2017) be 200% or larger. This measurement method is as described in Measurement Example 3 later. The elongation at break is a factor indicating the fracture resistance strength of a stent. In the case of a stent to be set in such an environment that plural kinds of physical actions such as bending, elongation, and pressing act on it due to a motion in a body cavity or a vascular duct, it is preferable that its elongation at break be 200% or larger. In the case of a stent to be set in a part where an even larger deformation may occur, it is preferable that the elongation at break of a stent be 500% or larger, even preferably 800% or larger, and particularly preferably 1000% or larger. It is preferable that the elongation at break of a stent may be as large as possible. Although there are no particular limitations on the upper limit of the elongation at break, a realistic upper limit of the elongation at break would be about 2500%.

Since the stent according to the embodiment is used being left in various body cavities and vascular ducts, the stent needs to have restorability (i.e., an ability to return to an original shape) even if the stent is deformed by receiving any of plural kinds of physical actions such as bending, elongation, and pressing due to a movement of the inside surface of a body cavity or a vascular duct. The restorability is a characteristic that is also necessary to allow a stent to be restored to an original state and fixed firmly to the wall of a vascular duct upon exit from the outlet of a trocar at a carriage destination after being inserted, in a deformed state, into the trocar or a catheter for carrying the stent and being carried. To this end, in the stent according to the embodiment, it is important that the restorability that is defined by the following formula be 40% or higher. Restorability can be evaluated quantitatively according to the following formula as will be described later in Measurement Example 3:

$$restorability ( \% ) = \{ ( {L}_{0} \times 2 - {L}_{1} ) / {L}_{0} \} \times 100$$

in which
L₀ is an initial length; and
L₁ is a length that is obtained after a manipulation of applying tensile stress to the stent in its longest direction so as to cause tensile strain of 100% based on the initial length L₀ has been performed ten times repeatedly.

As the restorability of a stent is closer to 100%, the stent is less prone to lose a function to be attained thereby due to its deformation. Since a stent receives plural kinds of physical actions such as bending, elongation, and pressing due to a movement of the inside surface of a body cavity or a vascular duct, it is preferable that the restorability of the stent according to the embodiment be 40% or higher, even preferably 50% or higher, further preferably 70% or higher, still preferably 75% or higher, especially preferably 80% or higher, and particularly preferably 85% or higher. It is preferable that the restorability of the stent be as high as possible; the upper limit of the restorability is 100%.

In addition, since the stent according to the embodiment is used being left in various body cavities and vascular ducts, the stent is preferably capable of suppressing sticking of mucus. Therefore, it is preferable that the stent according to the embodiment has a mucus sticking amount (%) defined by the following formula of 60% or less. The mucus sticking amount can be quantitatively evaluated by the following formula as discussed in Measurement Example 5 below.

$$Mucus sticking amount ( \% ) = ( As - Asb ) \times 100 / ( Ac - Acb )$$

As: absorbance at 450 nm of a sample
Asb: absorbance at 450 nm of a blank solution for the sample (PBS is used instead of a mucin solution and incubated for one night)
Ac: absorbance at 450 nm of a stent made of polylactic acid
Acb: absorbance at 450 nm of a blank solution for the stent made of polylactic acid (PBS is used instead of a mucin solution and incubated for one night)

As the mucus sticking amount of a stent is closer to 0%, the stent can be suppressed from sticking of mucus. Since sticking of mucus may cause a complication such as constriction of a stent or an infection, the stent according to the embodiment preferably has the mucus sticking amount of 60% or less and preferably 50% or less.

The stent according to the embodiment can be manufactured (formed or molded) using such bioabsorbable polyester copolymer by a melt molding method, a solvent molding/forming method, an electrospinning method, or a forming method using a 3D printer.

The melt molding method is a method that a polymer is heated and melted, and molded using a mold, an extrusion molding machine, a press machine, or the like. A stent can be manufactured by molding into a fiber shape, a film shape, a tube shape, or the like. For example, a thread-shaped polymer can be molded by heating a copolymer described in this specification to 200° C. in an extrusion molding machine in which a die of 1 mm in diameter is set and extruding it. A stent can be produced by weaving or knitting fibers thus formed.

The solvent molding/forming method is a method of performing molding by dissolving a polymer in a solvent, injecting a resulting solution into a mold or a coagulating bath, and separating the solvent and the solute from each other, and can manufacture a stent by molding a polymer into a fiber shape, a film shape, a tube shape, or the like. In a specific example of the solvent forming method, a rod of 0.5 to 20 mm in diameter is immersed in a polymer solution in which a polymer is dissolved in chloroform at a concentration 20%, the rod is pulled up from the solution, and the rod is immersed again after volatilization of the solvent. This cycle is repeated about five to 50 times and the core rod is pulled out finally, and thus a tubular stent can be formed.

The electrospinning method is a technique capable of forming a fiber structure made up of nanofibers of several nanometers in diameter by applying a high voltage to a polymer solution in a spinning nozzle, and can adjust the thickness of a fiber structure in a desired range by adjusting a spinning time. For example, a tubular fiber structure can be formed by collecting polymer fiber while rotating a cylindrical collector and then pulling out the collector from the fiber structure.

Furthermore, a tailor-made stent can be manufactured by employing bioabsorbable polyester copolymer as described above as an ink material for a 3D printer. The disclosure also provides manufacturing method of a stent including a step of performing 3D printing using a printing material containing bioabsorbable polyester copolymer as described above.

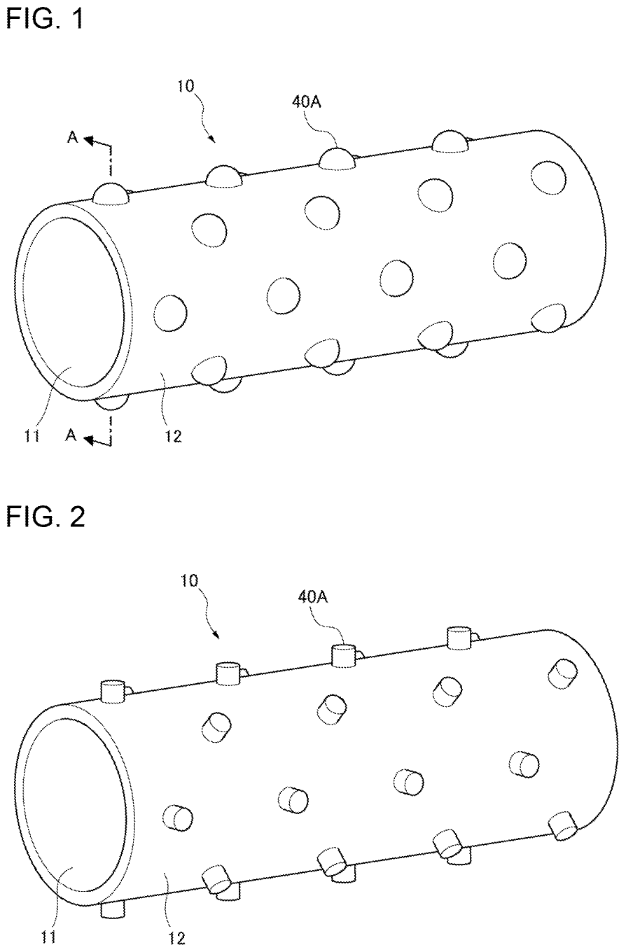

A description will be made of shapes of stents according to the embodiment. FIGS. 1 to 3 are schematic perspective views of stents according to specific examples of the embodiment.

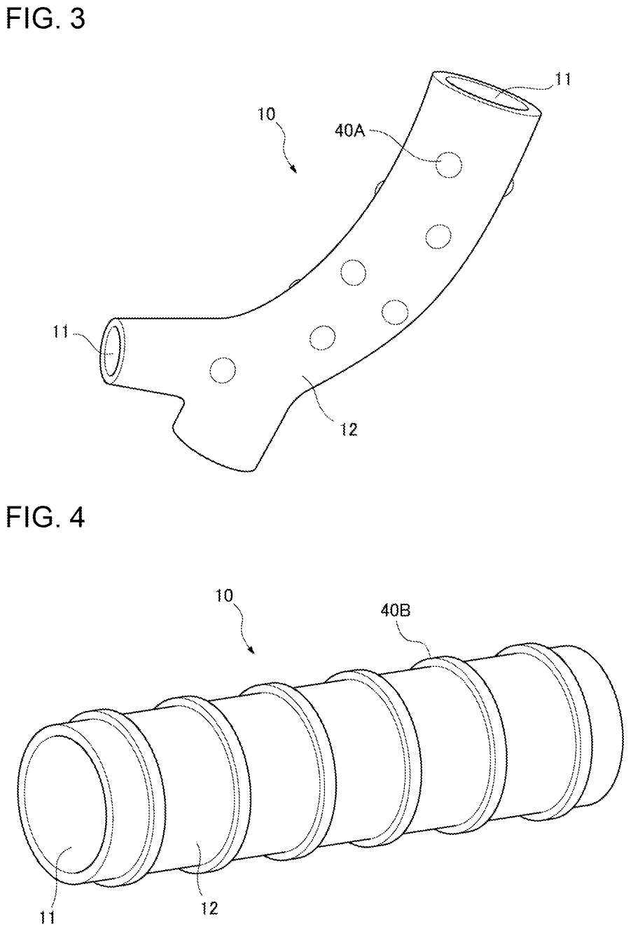

Although there are no particular limitations on the shape of the stent according to the embodiment, the stent may include a tubular structure portion as shown in FIG. 1. In the stent 10 shown in FIG. 1, the inside of the tubular structure portion is an inside surface 11 and the surfaces other than the inside surface 11 are outside surfaces 12. The tubular structure portion may be able to be expanded in the radial direction. FIG. 3 shows another stent according to the embodiment which have branches. It is preferable that the stent according to the embodiment have a shape that conforms to the shape of a respiratory organ to which the stent is to be applied.

Although there are no particular limitations on the size of the stent according to the embodiment, to serve as a stent for a respiratory organ, it is preferable that the tubular structure portion be 4 mm or larger and 24 mm or smaller in outer diameter and 0.2 mm or larger and 2 mm or smaller in thickness, even preferably 6 mm or larger and smaller than or equal to 20 mm in outer diameter and 0.25 mm or larger and 1.5 mm or smaller in thickness and further preferably 6 mm or larger and 20 mm or smaller in outer diameter and 0.3 mm or larger and 1.2 mm or smaller in thickness.

A safer stent that is low in the probability of causing a complication can be provided by controlling the thickness within the above-described range. A thickness of a stent can be calculated according to a formula ((stent outer diameter)−(stent inner diameter)}/2.

The term “outer diameter” as used herein is defined so that projections or projections/recesses are included in the case where they are formed on the outer circumferential surface. In the case where no projections or no projections/recesses are formed on the outer circumferential surface, the “outer diameter” is defined so that no projections or no projections/recesses are included and it is sufficient that a portion whose outer diameter is within the above range exist in a part of the stent.

Although the shape of the stent according to the embodiment is not limited to the ones described above, it is preferable that projections or projections and recesses may be formed on the outside surface of a stent to prevent the stent from moving after being set. It is preferable that plural projections or plural projections and recesses be formed.

Plural projections or projections and recesses may be arranged either regularly or randomly. In the case where projections are formed on the outside surface of a stent, plural projections 40A may be arranged regularly or randomly as shown in FIG. 1, 2, or 3, for example.

Projections or projections and recesses may be arranged locally on the outside surface, be arranged on the entire outside surface, or dot the outside surface.

There are no particular limitations on the shape of the projections 40A; each of them may be shaped like a semisphere, a cylinder, a cone, a prism, a polygonal spindle, a hook, or the like. More specifically, for example, each of the projections 40A may be shaped like a semisphere as shown in FIG. 1 or a cylinder as shown in FIG. 2.

There are no particular limitations on the shape of the projections/recesses; they may be shaped like folds, embossed shapes, certain patterns (e.g., lines, waves) or the like. More specifically, for example, the projections/recesses may be shaped like folds as shown in FIG. 6 or line-shaped patterns like projections 40B of a stent shown in FIG. 4.

In the case where projections or projections/recesses are arranged on the outside surface of the stent, there are no particular limitations on the size of the projections. From the viewpoint of inhibiting stimuli to tissue, the size (height) of the projections or projections/recesses is preferably 4.0 mm or shorter, even preferably 3.0 mm or shorter and further preferably 2.0 mm or shorter. To allow the stent to exhibit the function of preventing movement of itself after the stent is set and left, the size (height) of the projections or projections/recesses is preferably 0.1 mm or larger, even preferably 0.2 mm or larger.

For example, to prevent movement of the stent after being set and left, a stent having a size and a shape that are suitable for the shape of a respiratory organ of a patient to which the stent is to be applied may be produced by generating data being high in anatomical accuracy by 3D-CT and using a 3D printing technique on the basis of an anatomical analysis. The difference between the outer diameter of the stent and the inner diameter of a respiratory organ to which the stent is to be applied is preferably 10% or smaller, even preferably 8% or smaller, further preferably 6% or smaller, and particularly preferably 5% or smaller.

There are no particular limitations on the composition of the stent according to the embodiment except that it contains a bioabsorbable polyester copolymer as described above; either the entire stent or only part of the stent may be made of a bioabsorbable polyester copolymer.

Furthermore, the stent according to the embodiment may include a substrate and may include a resin layer on the surface of the substrate.

### [Substrate]

There are no particular limitations on the material of the substrate (stent substrate) of a stent; it may contain a metal or a resin.

Examples of the metal include stainless steel, a cobalt alloy, a titanium alloy, a nickel-titanium alloy (Nitinol).

Examples of the resin include polyurethane, polyester, bioabsorbable polyester copolymer, PTFE (polytetrafluoroethylene), and silicone resin. From the viewpoints of biocompatibility, kinetic properties, workability, etc., bioabsorbable polyester copolymer and silicone resin are preferable.

In the case where the materials of the substrate include bioabsorbable polyester copolymer, the substrate may be either one the whole of which is made of bioabsorbable polyester copolymer or one only part of which contains bioabsorbable polyester copolymer.

Examples of bioabsorbable polyester copolymer contained in the substrate include ones described above, and preferable examples of bioabsorbable polyester copolymer contained in the substrate are also ones described above.

The substrate may be made of either one kind of material or two or more kinds of materials.

### [Resin Layer]

The stent according to the embodiment may include a resin layer as a surface layer. The resin layer in a surface layer of a stent is formed by a resin formed on the surface of the substrate.

In the case where the stent according to the embodiment includes a resin layer, it is preferable that a mixed layer that is a mixture of a component of the substrate and a component of the resin layer be formed between the substrate and the resin layer.

Examples of the resin as a material of the resin layer include polyurethane, polyester, bioabsorbable polyester copolymer, PTFE (polytetrafluoroethylene), and a water-soluble polymer, among which bioabsorbable polyester copolymer and a water-soluble polymer are preferable.

In the case where the materials of the resin layer include bioabsorbable polyester copolymer, the resin layer may be either one the whole of which is made of bioabsorbable polyester copolymer or one only part of which contains bioabsorbable polyester copolymer.

It is preferable that the stent according to the embodiment include a resin layer containing a resin and contain 50 mass % or more of bioabsorbable polyester copolymer in the resin layer (100 mass %). The content of the bioabsorbable polyester copolymer is even preferably 70% or more, further preferably 95% or more, and particularly preferably 100 mass %, based on 100 mass % of the resin layer.

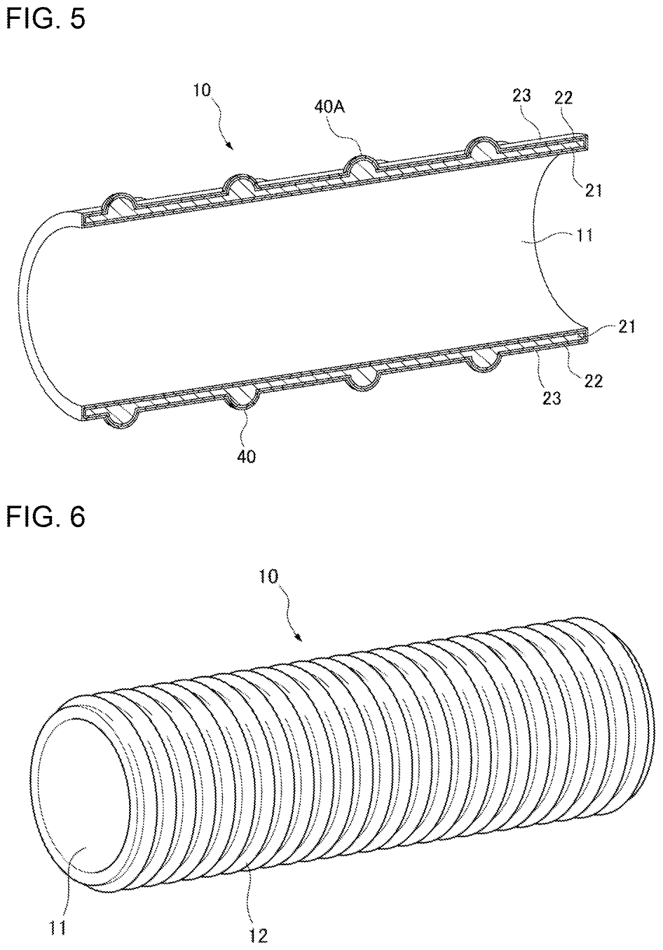

FIG. 5 is a sectional view, taken along line A-A, of the stent 10 shown in FIG. 1 according to one specific example of the embodiment. As shown in FIG. 5, it is preferable that the stent 10 include, between a substrate 21 and a resin layer 23, a mixed layer 22 that is a mixture of a component of the substrate 21 and a component of the resin layer 23.

The mixed layer which is formed between the substrate and the resin layer may be formed in such a manner that part of resin to constitute the resin layer goes into the inside of the substrate. Alternatively, the mixed layer may be formed in such a manner that part of the material to constitute the substrate goes into the inside of the resin layer. In the case where the stent according to the embodiment includes a mixed layer, it has a resin-containing layer that has a layered structure consisting of two or more layers including the resin layer and the mixed layer.

It is preferable that the stent according to the embodiment include a resin layer on at least part of the inside surface 11; a resin layer may be formed on the entire inside surface 11. The stent according to the embodiment may further include a resin layer on either part or all of the outside surface 12.

From the viewpoint of biocompatibility, it is preferable that the stent according to the embodiment include a resin layer on the entire inside surface 11 and outside surface 12, that is, on the entire surfaces of the stent.

The descriptions made above of bioabsorbable polyester copolymer and the water-soluble polymer employed can be applied as they are to bioabsorbable polyester copolymer and water-soluble polymer to constitute the resin layer.

The resin layer may be made of a single kind of material or two or more kinds of materials.

The stent according to the embodiment can be used a drug-eluting stent by allowing the stent to carry or absorb a drug.

Although the stent according to the embodiment can also be applied to uses for securing a lumen by implanting it into a constricted part of various body cavities or vascular ducts (vascular system, esophagus, gastrointestinal tract, colon and small intestine, bile duct, pancreatic duct, lung pipes, ureter, nasal cavities and trachea, etc.), its uses are not limited to them. Since the stent according to the embodiment is high in restorability because bioabsorbable polyester copolymer is contained and is able to be used as a drug-eluting stent, the stent according to the embodiment can be used suitably as a stent to be set in a vascular system, a trachea, a nasal tract, or the like. Further capable of suppressing sticking of mucus, the stent according to the embodiment can be used particularly suitably as a stent to be set in a trachea, a nasal tract, or the like.

### (Method for Securing Air Flow by Relieving Stenosis of Respiratory Organ)

The method according to the embodiment for securing air flow by relieving stenosis of a respiratory organ is a method for securing air flow by relieving stenosis of a respiratory organ using a stent for a respiratory organ, in which:
the stent contains the bioabsorbable polyester copolymer of the above-described embodiment.

By employing the above-described stent, the method according to the embodiment for securing air flow by relieving stenosis of a respiratory organ can inhibit sticking of mucus, is high in biocompatibility, and can inhibit occurrence of a complication.

The above description of the stent can be used as it is for the method according to the embodiment for securing air flow by relieving stenosis of a respiratory organ.

### INDUSTRIAL APPLICABILITY

The stent according to the embodiment is a stent that can be applied to respiratory organs; for example, it can be applied to the respiratory tract, oral cavity, nasal tracts, pharynx, trachea, bronchi, bronchioles, and lungs. The stent according to the embodiment can also be applied to uses for securing a lumen by implanting it into a constricted part of various body cavities or vascular ducts (vascular system, esophagus, gastrointestinal tract, colon and small intestine, bile duct, pancreatic duct, ureter, etc.) other than the respiratory organs.

### EXAMPLES

The disclosure will be described below in detail using Examples and Comparative Examples. However, the disclosure is not limited to them.

### (Measurement Example 1: Measurement of Mole Fractions of Respective Kinds of Residues and an R Value by Nuclear Magnetic Resonance (NMR))

A refined polyester copolymer was dissolved in deuterochloroform and proportions of lactic acid monomer residues and caprolactone monomer residues in the polyester copolymer were calculated through a measurement by ¹H-NMR. A ¹H homospin decoupling method was employed to separate combinations of two adjacent monomer residues from each other using a signal indicating whether lactic acid or caprolactone was adjacent to a monomer residue for the methine group (around 5.10 ppm) of lactic acid and the α methylene group (around 2.35 ppm) and the E methylene group (around 4.10 ppm) of caprolactone and respective peak areas were quantified. Also, in the case where δ-valerolactone was used instead of ε-caprolactone, combinations of two adjacent monomer residues were separated from each other using a signal indicating whether lactic acid or valerolactone was adjacent to a monomer residue for the methine group (around 5.10 ppm) of lactic acid and the α methylene group (around 2.35 ppm) and the ε methylene group (around 4.10 ppm) of valerolactone and respective peak areas were quantified.

[AB] and an R value were calculated from the thus-obtained peak area ratios. [AB] is a mole fraction of structures in which a lactic acid residue and a caprolactone residue or valerolactone residue are adjacent to each other in the polyester copolymer. More specifically, [AB] is a ratio (%) of the sum of the number of A-B dyads and number of B-A dyads to the total number of A-A dyads, A-B dyads, B-A dyads, and B-B dyads. Results are shown in Table 1.
Instrument name: JNM-ECZ400R (produced by JEOL Ltd.)
¹H homospin decoupling irradiation position: 1.66 ppm
Solvent: deuterochloroform
Measurement temperature: room temperature (20° C. to 25° C.)

$$R = [ AB ] / ( 2 [ A ] [ B ] ) \times 100$$

in which:
[A] is a mole fraction (%) of monomer A residues in the polyester copolymer;
[B] is a mole fraction (%) of monomer B residues in the polyester copolymer; and
[AB] is a mole fraction (%) of structures (A-B and B-A) in which a monomer A residue and a monomer B residue are adjacent to each other in the polyester copolymer.

### (Measurement Example 2: Measurement of a Weight-Average Molecular Weight by Gel-Permeation Chromatography (GPC))

A weight-average molecular weight of a polyester copolymer used was measured under the following conditions:

### (GPC Measurement Conditions)

Instrument name: Prominence (produced by Shimadzu Corporation)
Mobile phase: chloroform (for HPLC) (produced by Wako Pure Chemical Corporation)
Flow rate: 1 mL/min
Column: TSKgel GMHHR-M (7.8 mm (diameter)×300 mm, produced by Tosoh Corporation)
Detector: UV (254 mm), RI
Column, detector temperature: 35° C.
Standard substance: polystyrene

A weight-average molecular weight of polyester copolymer was calculated by dissolving a refined polyester copolymer in chloroform, eliminating impurities by passing a resulting solution through a 0.45-μm syringe (DISMIC-13HP produced by Advantec Co., Ltd.), and subjecting a resulting solution to a measurement by GPC. Results are shown in Table 1.

### Measurement Example 3: Tensile Test

A test piece measuring 40 mm×5 mm was cut out from a stent (thickness: 1.0 mm) manufactured in each Example or Comparative Example and subjected to a tensile test under the following conditions according to JIS K6251 (2017) using a Tensilon universal tester RTM-100 (produced by Orientec Co., Ltd.), and elongation at break and tensile strength were calculated. Furthermore, a slope of an approximate linear equation obtained from data of five points, obtained from a start of application of stress, in a stress-strain graph was calculated as a Young's modulus.

When necessary, two gauge lines were drawn on the test piece using a proper marker. Accurate and clear gauge lines were drawn perpendicularly to parallel portions of the test piece at positions that are equally distant from the center of the test piece in a state that the test piece is not pulled.
Instrument name: EZ-1kNLX (produced by Shimadzu Access Corporation)
Distance between gauge lines before test: 10 mm
Distance between gripping tools: 10 mm (gripping at the gauge line positions)
Pulling rate: 500 mm/min
Load cell: 1 kN
Number of times of testing: 5 times

As for restorability, tensile strain of 100% with respect to the distance between the gripping tools before the test was caused at a pulling rate of 500 mm/min (manipulation 1). Immediately after manipulation 1 (shape holding time: 0 sec), the tensile strain was relaxed at a rate of 500 mm/min and the distance between the gripping tools was returned to 5 mm (manipulation 2). Immediately after manipulation 2 (shape holding time: 0 sec), manipulation 1 and manipulation 2 were performed again. After this cycle was performed repeatedly so that manipulation 1 and manipulation 2 were performed 10 times respectively, restorability was calculated according to the following formula using a value L₁ obtained:

$$( restorability ( \% ) ) = \{ ( {L}_{0} \times 2 - {L}_{1} ) / {L}_{0} \} \times 100$$

in which:
L₀ is initial length (the distance between the gauge lines before the test); and
L₁ is a length (a distance between the gauge lines after the test) that was obtained after a manipulation of applying tensile stress to the stent in its longest direction so as to cause tensile strain of 100% with respect to the initial length L₀ was performed ten times repeatedly.

### Measurement Example 4: Evaluation of Water Solubility of Polymer

1 g of a polymer was put into 100 mL of water at 37° C. and a resulting solution was stirred for 3 hours. After that, the solution was checked visually. If the polymer was dissolved, the polymer was judged water-soluble.

### Measurement Example 5: In Vitro Mucus Sticking Test

Mucin was purified from saliva and a mucin solution having a concentration of 100 μg/mL was prepared. A stent was punched into disc-shaped pieces having a diameter of 4 mm, and then the obtained pieces were set in 48 respective wells of a microtiter plate. To each well, 600 μL of the mucin solution having a concentration of 100 μg/mL was added and incubated at 37° C. for 20-24 hours. As a control, PBS was added instead of a mucin solution and incubated at 37° C. for 20-24 hours. After cleaning was performed three times using PBS, a blocking buffer (ThermoFisher Scientific 37570) was added and incubated at room temperature (20° C. to 25° C.) for one hour. After cleaning was performed three times using PBS, WGA (Biotinylated Wheat Germ Agglutinin (WGA), Vector Laboratories B-1025-5; diluted 500 times by PBS) was added and incubated at room temperature for one hour. After cleaning was performed three times using PBS, horseradish peroxidase (H1RP)-conjugated streptavidin (HRP-Streptavidin, Sigma-Aldrich RABRP3-600UL) was added and incubated at room temperature for one hour. After cleaning was performed three times using PBS, 250 μL of a solution of TMB (3,3′,5,5′-tetramethylbenzidine (TMB) substrate, Thermo Scientific P134028) was added and incubated at room temperature for 15 to 30 minutes. Then each sample was taken out, 250 μL of 2 mol/L sulfuric acid was added and absorbance at 450 nm was measured using a microplate spectrophotometer. A mucus (mucin) sticking amount was calculated according to the following formula (1).

### [Mucus Sticking Amount (%)]

$$( Mucus sticking amount ( \% ) ) = ( As - Asb ) \times 100 / ( Ac - Acb ) \qquad Formula ( 1 )$$

As: absorbance at 450 nm of a sample
Asb: absorbance at 450 nm of a blank solution for the sample (PBS is used instead of a mucin solution and incubated for one night)
Ac: absorbance at 450 nm of a stent made of polylactic acid
Acb: absorbance at 450 nm of a blank solution for the stent made of polylactic acid (PBS is used instead of a mucin solution and incubated for one night)

### [Phosphorate Buffer Solution]

The composition of the phosphorate buffer solution was as follows:
KCl: 0.2 g/L
KH₂PO₄: 0.2 g/L
NaCl: 8.0 g/L
Na₂HPO₄ (anhydrous): 1.15 g/L
EDTA: 0.25 g/L.

### Example 1

50.0 g of L-lactide (PURASORB L produced by Purac Biochem B.V.) and 39.6 g of ε-caprolactone (produced by FUJIFILM Wako Pure Chemical Corporation) as monomers and 0.46 g of hydroxypivalic acid as an initiator were put into a separable flask. In an argon atmosphere, 0.27 g of tin(II) octylate (produced by FUJIFILM Wako Pure Chemical Corporation) as a catalyst dissolved in 5.8 mL of toluene (super dehydrated, produced by FUJIFILM Wako Pure Chemical Corporation) was added to them and reaction was caused at 140° C. for 9.5 hours, and thus a coarse copolymer was obtained.

The thus-obtained coarse copolymer was dissolved in 200 mL of chloroform and a resulting solution was dripped into 3000 mL of hexane in a stirred state, and precipitates were obtained. The precipitates were dried under reduced pressure at 50° C. and thus a macromer was obtained.

50 g of the macromer, 2.9 g of 4-(dimethylamino)pyridinium-p-toluenesulfonate (synthesized) as a catalyst, and 1.2 g of 4,4-dimethylaminopyridine (produced by FUJIFILM Wako Pure Chemical Corporation) were taken. In an argon atmosphere, they were dissolved in 200 mL of dichloromethane (dehydrated, produced by FUJIFILM Wako Pure Chemical Corporation), 2.4 mL of diisopropylcarbodiimide (produced by FUJIFILM Wako Pure Chemical Corporation) as a condensing agent was added, and condensation polymerization was caused at room temperature (20° C. to 25° C.) for 20 hours.

A resulting reaction mixture was diluted in 220 mL of chloroform, 10 g of lactic acid was added, and a resulting solution was stirred for 3 hours. Then 330 mL of ion-exchanged water was added, a resulting solution was stirred for 15 minutes, and a step of eliminating a water phase by decantation was repeated until the pH of a removed water phase became equal to 7. A remaining organic phase was dripped into 2200 mL of methanol in a stirred state and precipitates were obtained. The precipitates were dried under reduced pressure at 50° C., and thus a refined polyester copolymer of Example 1 was obtained.

The thus-obtained polyester copolymer was cut into cubes whose edges measured about 5 mm and they were put into an extruder. The polyester copolymer was extruded at a set temperature 100° C. to 200° C. so that a resulting filament had a diameter of 1.75 mm. Using the filament, a stent that was a tubular formed body as shown in FIG. 6 (inner diameter: 10 mm, thickness: 1.0 mm, and length: 40 mm) was produced using a fused deposition modeling-type 3D printer.

### Example 2

60.0 g of L-lactide (PURASORB L produced by Purac Biochem B.V.) and 31.7 g of ε-caprolactone (produced by FUJIFILM Wako Pure Chemical Corporation) as monomers and 0.46 g of hydroxypivalic acid as an initiator were put into a separable flask. In an argon atmosphere, 0.27 g of tin(II) octylate (produced by FUJIFILM Wako Pure Chemical Corporation) as a catalyst dissolved in 5.8 mL of toluene (super dehydrated, produced by FUJIFILM Wako Pure Chemical Corporation) was added to them and reaction was caused at 140° C. for 9.5 hours, and thus a coarse copolymer was obtained.

The thus-obtained coarse copolymer was dissolved in 200 mL of chloroform and a resulting solution was dripped into 3000 mL of hexane in a stirred state, and precipitates were obtained. The precipitates were dried under reduced pressure at 50° C. and thus a macromer was obtained.

50 g of the macromer, 2.1 g of 4-(dimethylamino)pyridinium-p-toluenesulfonate (synthesized) as a catalyst, and 0.87 g of 4,4-dimethylaminopyridine (produced by FUJIFILM Wako Pure Chemical Corporation) were taken. In an argon atmosphere, they were dissolved in 200 mL of dichloromethane (dehydrated, produced by FUJIFILM Wako Pure Chemical Corporation), 1.7 mL of diisopropylcarbodiimide (produced by FUJIFILM Wako Pure Chemical Corporation) as a condensing agent was added, and condensation polymerization was caused at room temperature (20° C. to 25° C.) for 20 hours.

A resulting reaction mixture was diluted in 220 mL of chloroform, 10 g of lactic acid was added, and a resulting solution was stirred for 3 hours. Then 330 mL of ion-exchanged water was added, a resulting solution was stirred for 15 minutes, and a step of eliminating a water phase by decantation was repeated until the pH of a removed water phase became equal to 7. A remaining organic phase was dripped into 2200 mL of methanol in a stirred state and precipitates were obtained. The precipitates were dried under reduced pressure at 50° C., and thus a refined polyester copolymer of Example 2 was obtained.

A stent that was a tubular formed body was produced in the same manner as in Example 1.

### Example 3

A refined polyester copolymer of Example 3 was obtained by the same method as in Example 1 except that the amount of hydroxypivalic acid was changed to 0.45 g, the reaction temperature for obtaining coarse copolymer was changed to 150° C., the amount of 4-(dimethylamino)pyridinium-p-toluenesulfonate was changed to 2.1 g, the amount of 4,4-dimethylaminopyridine was changed to 0.87 g, and the amount of diisopropylcarbodiimide was changed to 1.7 mL.

A stent that was a tubular formed body was produced in the same manner as in Example 1.

### Example 4

50.0 g of L-lactide (PURASORB L produced by Purac Biochem B.V.) and 38.5 g of ε-caprolactone (produced by Wako Pure Chemical Corporation) as monomers were put into a separable flask. In an argon atmosphere, 0.81 g of tin(II) octylate (produced by Wako Pure Chemical Corporation) as a catalyst dissolved in 14.5 mL of toluene (super dehydrated, produced by Wako Pure Chemical Corporation) and ion-exchanged water as an auxiliary initiator were added to them so that the monomer/auxiliary initiator ratio became 142.9, auxiliary catalyzation reaction was caused at 90° C. for 1 hour, and copolymerization reaction was caused at 150° C. for 6 hours, and thus a coarse copolymer was obtained.

The thus-obtained coarse copolymer was dissolved in 100 mL of chloroform and a resulting solution was dripped into 1400 mL of methanol in a stirred state, and precipitates were obtained. After this manipulation was repeated three times, resulting precipitates were dried under reduced pressure at 70° C., and thus a macromer was obtained.

30 g of the macromer, 0.28 g of 4-(dimethylamino)pyridinium-p-toluenesulfonate (synthesized) as a catalyst, and 0.10 g of 4,4-dimethylaminopyridine (produced by Wako Pure Chemical Corporation) were taken. In an argon atmosphere, they were dissolved in dichloromethane (dehydrated, produced by Wako Pure Chemical Corporation) so that the concentration became 30%, 0.47 g of amylene (produced by Tokyo Chemical Industry Co., Ltd.) as a condensing agent dissolved in 5 mL of dichloromethane was added, and condensation polymerization was caused at room temperature (20° C. to 25° C.) for two days.

30 mL of chloroform was added to a resulting reaction mixture and a resulting solution was dripped into 500 mL of methanol in a stirred state, and precipitates were obtained. The precipitates were dissolved in 50 mL of chloroform and a resulting solution was dripped into 500 mL of methanol in a stirred state, and precipitates were obtained. After this manipulation was repeated two times and resulting precipitates were dried under reduced pressure at 50° C., and thus a refined polyester copolymer of Example 4 was obtained. A stent that was a tubular formed body was produced in the same manner as in Example 1.

### Example 5

45 g of the polyester copolymer obtained in Example 3 and 5 g of polylactic acid (produced by Nature3D) were dissolved in 500 mL of chloroform (produced by FUJIFILM Wako Pure Chemical Corporation) and a resulting solution was added to 3000 mL of hexane in a stirred state. Resulting precipitates were dried at room temperature (20° C. to 25° C.) under normal pressure for 24 hours. Then the precipitates were dried under reduced pressure at 50° C., and thus a polymer composition of Example 5 was obtained. A stent that was a tubular formed body was produced in the same manner as in Example 1.

### Example 6

A polymer composition of Example 6 was obtained by performing manipulations by the same method as in Example 5 except that the amount of the polyester copolymer was changed to 35 g and the amount of polylactic acid was changed to 15 g. A stent that was a tubular formed body was produced in the same manner as in Example 1.

### Example 7

48.5 g of the polyester copolymer obtained in Example 1 and 1.5 g of polyethyleneglycol (produced by Sigma-Aldrich Co. LLC) were dissolved in 500 mL of chloroform (produced by FUJIFILM Wako Pure Chemical Corporation) and a resulting solution was added to 3000 mL of hexane in a stirred state and precipitates were obtained. The precipitates were dried at room temperature (20° C. to 25° C.) under normal pressure for 24 hours. Then the precipitates were dried under reduced pressure at 50° C., and thus a polymer composition of Example 7 was obtained. A stent that was a tubular formed body was produced in the same manner as in Example 1.

### Example 8

A polymer composition of Example 8 was obtained by performing manipulations by the same method as in Example 7 except that the amount of the polyester copolymer obtained in Example 1 was changed to 48 g and the amount of polyethyleneglycol was changed to 2.0 g. A stent that was a tubular formed body was produced in the same manner as in Example 1.

### Example 9

A polymer composition of Example 9 was obtained by performing manipulations by the same method as in Example 7 except that the amount of the polyester copolymer obtained in Example 1 was changed to 47.5 g and the amount of polyethyleneglycol was changed to 2.5 g.

A stent that was a tubular formed body was produced in the same manner as in Example 1.

### Example 10

A refined polyester copolymer of Example 10 was obtained by synthesizing it by the same method as in Example 1 except that the amount of 4-(dimethylamino)pyridinium-p-toluenesulfonate was changed to 1.5 g and the amount of diisopropylcarbodiimide was changed to 1.2 mL.

A stent that was a tubular formed body was produced in the same manner as in Example 1.

### Example 11

50.0 g of L-lactide (PURASORB L produced by Purac Biochem B.V.) as a monomer was put into a separable flask. In an argon atmosphere, 0.81 g of tin(II) octylate (produced by Wako Pure Chemical Corporation) as a catalyst dissolved in 14.5 mL of toluene (super dehydrated, produced by Wako Pure Chemical Corporation) was added to it and polymerization reaction was caused at 150° C. for 3 hours. Then 38.5 mL of ε-caprolactone (produced by Wako Pure Chemical Corporation) was added to them and polymerization reaction was caused at 150° C. for 6 hours, and thus a coarse copolymer was obtained.

The thus-obtained coarse copolymer was dissolved in 100 mL of chloroform and a resulting solution was dripped into 1400 mL of methanol in a stirred state, and precipitates were obtained. After this manipulation was repeated three times, precipitates were dried under reduced pressure at 70° C., and thus refined polyester copolymer of Example 11 was obtained.

A stent that was a tubular formed body was produced in the same manner as in Example 1.

### Comparative Example 1

Polylactic acid (produced by Nature3D) was cut into cubes whose edges measured about 5 mm and they were put into an extruder. The polyester copolymer was extruded at a set temperature 100° C. to 200° C. so that a resulting filament had a diameter of 1.75 mm. Using the filament, a stent that was a tubular formed body was obtained in the same manner as in Example 1.

Various measurement results of the polymers and stents obtained in Examples 1 to 11 and Comparative Example 1 are shown in Table 1.

TABLE 1

| | | Ex. 1 | Ex. 2 | Ex. 3 | Ex. 4 | Ex. 5 | Ex. 6 |
|---|---|---|---|---|---|---|---|
| Monomer A residue | | 52 | 56 | 51 | 51 | 51 | 51 |
| ratio (mol %) | | | | | | | |
| R value | | 0.60 | 0.71 | 0.82 | 0.51 | 0.82 | 0.82 |
| Bioabsorbable | Weight-average | 249.225 | 203.711 | 266.452 | 368.512 | 266.452 | 266.452 |
| polyester | molecular weight | | | | | | |
| (polyester | Polymer | Link | Link | Link | Link | Link | Link |
| copolymer) | structure | structure | structure | structure | structure | structure | structure |
| | V_{X}/V_{Y} | 4.1 | 4.1 | 4.1 | 4.1 | 4.1 | 4.1 |
| | Ratio in stent | 100 | 100 | 100 | 100 | 90 | 70 |
| | (mass %) | | | | | | |
| Bioabsorbable | Monomer | — | — | — | — | Lactic | Lactic |
| polyester | | | | | | acid | acid |
| (homopolymer) | Biodegradability | — | — | — | — | Present | Present |
| | Ratio in stent | — | — | — | — | 10 | 30 |
| | (mass %) | | | | | | |
| Water-soluble | Monomer | — | — | — | — | — | — |
| polymer | Water solubility | — | — | — | — | — | — |
| | Ratio in stent | — | — | — | — | — | — |
| | (mass %) | | | | | | |
| Stent | Restorability | 85 | 82 | 87 | 89 | 86 | 83 |
| | Young's modulus | 3.4 | 2.6 | 3.3 | 2.8 | 3.2 | 5.8 |
| | (MPa) | | | | | | |
| | Tensile strength | 25 | 27 | 29 | 36 | 33 | 38 |
| | (MPa) | | | | | | |
| | Elongation at | 1016 | 1134 | 1096 | 1522 | 901 | 823 |
| | break (%) | | | | | | |
| | Mucin sticking | 33 | 35 | 38 | 32 | 43 | 49 |
| | amount (%) | | | | | | |
| | | | | | | | Comp. |
| | | Ex. 7 | Ex. 8 | Ex. 9 | Ex. 10 | Ex. 11 | Ex. 1 |
| Monomer A residue | | 52 | 52 | 52 | 52 | 55 | — |
| ratio (mol %) | | | | | | | |
| R value | | 0.60 | 0.60 | 0.60 | 0.82 | 0.29 | — |
| Bioabsorbable | Weight-average | 249.225 | 249.225 | 249.225 | 86.850 | 180.000 | — |
| polyester | molecular weight | | | | | | |
| (polyester | Polymer | Link | Link | Link | Link | Non-link | — |
| copolymer) | structure | structure | structure | structure | structure | structure | |
| | V_{X}/V_{Y} | 4.1 | 4.1 | 4.1 | 4.1 | 4.1 | — |
| | Ratio in stent | 97 | 96 | 95 | 100 | 100 | — |
| | (mass %) | | | | | | |
| Bioabsorbable | Monomer | — | — | — | — | — | Lactic |
| polyester | | | | | | | acid |
| (homopolymer) | Biodegradability | — | — | — | — | — | Present |
| | Ratio in stent | — | — | — | — | — | 100 |
| | (mass %) | | | | | | |
| Water-soluble | Monomer | Ethylene | Ethylene | Ethylene | — | — | — |
| polymer | | glycol | glycol | glycol | | | |
| | Water solubility | Present | Present | Present | — | — | — |
| | Ratio in stent | 3 | 4 | 5 | — | — | — |
| | (mass %) | | | | | | |
| Stent | Restorability | 86 | 84 | 85 | 41 | 58 | 9 |
| | Young's modulus | 3.5 | 3.2 | 3.2 | 6.8 | 43 | 1264 |
| | (MPa) | | | | | | |
| | Tensile strength | 24 | 22 | 23 | 4.8 | 13 | 341 |
| | (MPa) | | | | | | |
| | Elongation at | 1093 | 1176 | 1158 | 2011 | 491 | 171 |
| | break (%) | | | | | | |
| | Mucin sticking | 29 | 26 | 22 | 38 | 52 | 100 |
| | amount (%) | | | | | | |

Link structure: structure in which two or more macromer units are connected together
Non-link structure: structure made up of one macromer unit

The term “monomer A residue ratio” used in Table 1 means a mole fraction (mol %) of monomer A residues to the total mole number of monomer A residues and monomer B residues (100%). In Examples 1 to 11, monomer A is L-lactide and monomer B is ε-caprolactone. V_{X} is an initial polymerization rate of L-lactide and V_{Y} is an initial polymerization rate of ε-caprolactone.

### REFERENCE SIGNS LIST

10: Stent
11: Inside surface
12: Outside surface
21: Substrate
22: Mixed layer
23: Resin layer
40A: Projection
40B: Projection

1. A stent for a respiratory organ comprising a bioabsorbable polyester copolymer, wherein
the bioabsorbable polyester copolymer is a polyester copolymer having residues of two kinds of ester bond forming monomers as main constituent units; and
where the two kinds of ester bond forming monomers are referred to as monomer A and monomer B, respectively, an R value given by the following formula is 0.25 or larger and 0.99 or smaller:
$$R = [ AB ] / ( 2 [ A ] [ B ] ) \times 100$$
in which:
[A] is a mole fraction (%) of monomer A residues in the polyester copolymer;
[B] is a mole fraction (%) of monomer B residues in the polyester copolymer; and
[AB] is a mole fraction (%) of structures (A-B and B-A) in which a monomer A residue and a monomer B residue are adjacent to each other in the polyester copolymer.

2. The stent according to claim 1, having a Young's modulus as measured in accordance with JIS K6251 (2017) of 0.1 MPa or larger and 50 MPa or smaller.

3. The stent according to claim 1, having a restorability of 40% or higher, the restorability being defined by the following formula:
$$restorability ( \% ) = ( {L}_{0} \times 2 - {L}_{1} ) / {L}_{0} \times 100$$
in which:
L₀ is an initial length; and
L₁ is a length that is obtained after a manipulation of applying tensile stress to the stent in its longest direction so as to cause tensile strain of 100% based on the initial length L₀ has been performed ten times repeatedly.

4. The stent according to claim 1, having a mucus sticking amount of 60% or less, the mucus sticking amount being defined by the following formula:
$$mucus sticking amount ( \% ) = ( As - Asb ) \times 100 / ( Ac - Acb )$$
in which:
As is an absorbance at 450 nm of a sample;
Asb is an absorbance at 450 nm of a blank solution for the sample (PBS is used instead of a mucin solution and incubated for one night);
Ac is an absorbance at 450 nm of a stent made of polylactic acid; and
Acb is an absorbance at 450 nm of a blank solution for the stent made of polylactic acid (PBS is used instead of a mucin solution and incubated for one night).

5. The stent according to claim 1, further comprising a water-soluble polymer.

6. The stent according to claim 5, having a water-soluble polymer content of 0.1 mass % or more and 25 mass % or less, the water-soluble polymer content being defined by the following formula:
$$water - soluable polymer content ( mass \% ) = [ M 1 / ( M 1 + M 2 ) \times 100 ]$$
in which
M1 is a mass of the water-soluble polymer; and
M2 is a mass of the polyester copolymer.

7. The stent according to claim 5, wherein the water-soluble polymer is a polyalkylene glycol.

8. The stent according to claim 1, wherein the stent contains 50 mass % or more of the bioabsorbable polyester copolymer based on 100 mass % of the stent.

9. The stent according to claim 1, wherein:
the monomer A is at least one selected from the group consisting of lactic acid and glycolic acid; and
the monomer B is at least one selected from the group consisting of caprolactone and δ-valerolactone.

10. The stent according to claim 1, having an outer diameter of 4 mm or longer and 24 mm or shorter and a thickness of 0.2 mm or larger and 2 mm or smaller.

11. The stent according to claim 1, having a plurality of projections or a plurality of projections/recesses on an outside surface.

12. The stent according to claim 11, wherein the projections or the projections/recesses have a height of 0.1 mm or larger and 3.0 mm or shorter.

13. A method for manufacturing the stent according to claim 1, comprising performing 3D printing using a printing material containing the bioabsorbable polyester copolymer.

14. A method for securing air flow by relieving stenosis of a respiratory organ using a stent for a respiratory organ, wherein:
the stent comprises a bioabsorbable polyester copolymer,
the bioabsorbable polyester copolymer is a polyester copolymer having residues of two kinds of ester bond forming monomers as main constituent units; and
where the two kinds of ester bond forming monomers are referred to as monomer A and monomer B, respectively, an R value given by the following formula is 0.25 or larger and 0.99 or smaller:
$$R = [ AB ] / ( 2 [ A ] [ B ] ) \times 100$$
in which:
[A] is a mole fraction (%) of monomer A residues in the polyester copolymer;
[B] is a mole fraction (%) of monomer B residues in the polyester copolymer; and
[AB] is a mole fraction (%) of structures (A-B and B-A) in which a monomer A residue and a monomer B residue are adjacent to each other in the polyester copolymer.
